(19) United States
(12) Patent Application Publication
(10) Pub. No.: US 2023/0260620 A1
(43) Pub. Date: Aug. 17, 2023

(54) COMPUTERIZED SYSTEMS AND METHODS FOR DYNAMIC DETERMINATION AND APPLICATION OF ADJUSTED ELECTRONIC STIMULUS PATTERNS

(71) Applicant: Sana Health, Inc., Lafayette, CO (US)

(72) Inventor: Richard Hanbury, Sandy, UT (US)

(73) Assignee: SANA HEALTH, INC., Lafayette, CO (US)

(21) Appl. No.: 18/111,217

(22) Filed: Feb. 17, 2023

Related U.S. Application Data
(60) Provisional application No. 63/319,888, filed on Mar. 15, 2022.
(60) Provisional application No. 63/311,403, filed on Feb. 17, 2022.

(51) Int. Cl. G16H 20/30; G16H 10/60; G16H 50/20; A61N 1/36; A61B 5/00

(52) U.S. Cl. CPC G16H 20/30; G16H 10/60; G16H 50/20; A61N 1/36031; A61B 5/4848

(57) ABSTRACT

Disclosed are systems and methods for a computerized framework that detects medical conditions within patients and dynamically effectuates a treatment via a recursively trained machine learning (ML) algorithm. The disclosed framework is configured for controlling computerized equipment by analyzing data associated with a patient, determining underlying conditions of the patient, then automatically causing such equipment to output electronic stimuli that can address the medical condition(s) detected. The framework can determine a correlation between a patient's attributes, electronic data of a condition of a patient and a medical disorder, and cause a device (e.g., a neuromodulation device) to treat and monitor improvements of the condition and disorder. The treatments can be dynamically adjusted, controlled and monitored so that electronic stimulus patterns respective to a patient's conditions can be rendered.

### CROSS-REFERENCE TO RELATED APPLICATIONS

This application claims the benefit of priority from U.S. Provisional No. 63/319,888, filed on Mar. 15, 2022, entitled “Computerized Systems and Methods for Dynamic Determination and Application of Adjusted Electronic Stimulus Patterns,” and U.S. Provisional No. 63/311,403, filed on Feb. 17, 2022, entitled “Computerized Systems and Methods for Adaptive Machine Learning-Based Dynamic Adjusted Stimulus Pattern Detection,” which are commonly owned and incorporated herein in their entirety by reference.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### FIELD OF THE DISCLOSURE

The present disclosure relates to a diagnostic and therapeutic system for detecting and treating medical conditions of patients, and more particularly, towards dynamically adjusting and monitoring machine learning (ML) controlled electronic stimulus patterns respective to a patient for purposes of diagnosing and treating the patient's medical conditions.

### BACKGROUND

Of the many methodologies for detecting and treating disorders or medical conditions within patients, sensory stimulation can be used by medical professionals. Indeed, medical professionals typically rely on computer-related imagery to detect medical disorders, while relying on pharmaceuticals and/or other chemical supplements to address the underlying conditions.

For example, magnetic resonance imaging (MRI), electromagnetic waves (e.g., x-rays) and computed tomography (CT) technologies can capture imagery of a patient, which then must be analyzed by a medical professional (e.g., a doctor, nurse, or both). Based on this analysis, the medical professional prescribes a treatment, which typically involves medication (or in some cases, some form of physical stimulus—e.g., radiation for treating cancer, for example).

### SUMMARY

Unfortunately, such methodologies are not always effective, nor agile enough to adjust to the dynamic nature of many current medical conditions, as they suffer from human error, mis-prescribed treatments, and improperly taken doses by the patient, among other drawbacks. Moreover, the experimental nature of diagnosing and treating conditions is based on a “trail-and-error” formula, which involves changing prescribed medication doses should the conditions not be improving as expected (which usually takes weeks, if not months).

The disclosed framework addresses these shortcomings, among others, by providing a novel computerized framework that detects medical conditions within patients and dynamically treats them accordingly via a recursively trained (e.g., closed-loop) machine learning (ML) algorithm. According to some embodiments, the disclosed framework is configured for and capable of controlling computerized equipment by analyzing data associated with a patient, determining underlying conditions of the patient, then automatically causing such equipment to output electronic stimuli that can address (e.g., effectively treat with the purpose of curing) the medical condition(s). Embodiments of the disclosed framework, including environments where the disclosed ML algorithm can operate, which sub-modules it contains, and how it operates are discussed below in relation to engine 200 and FIGS. 1-6.

By way of a non-limiting example, the disclosed systems and methods can operate to receive signal data from brain imaging of a patient. The disclosed framework can operate to analyze the data and detect therefrom conditions associated with a medical disorder (e.g., insomnia) of the patient. Rather than simply prescribing medication, such as, for example AMBIEN®, the disclosed systems and methods can operate to analyze the condition, determine a form of electronic stimulus to provide to the patient, and then effectuate a treatment that includes the determined stimulus via connected devices to the patient that can deliver the prescribed stimulus. As discussed herein, the characteristics (e.g., type and quantity, for example) of the stimulus are determined via a recursive learning operation (e.g., engine 200, as discussed below) that enables the framework to determine which treatments operate best for particular patients based on the patient's condition and other underlying factors (e.g., demographics, biometric information, medical history, geographic information and other profile or Electronic Medical Record (EMR) related data of the patient, and the like), as discussed below in more detail.

As evident from the below discussion, the disclosed systems and methods provide computerized capabilities that can be modeled for i) determining a correlation between attributes of a patient (e.g., age, for example, and/or other EMR information and/or demographics) and electronic data of a condition of a patient (e.g., slow wave sleep) and a medical disorder (e.g., dementia—where a reduction in slow wave sleep is associated with an increase in the incidence of dementia), ii) determining whether improving the condition (e.g., providing stimuli to increase the wave sleep) also improves the medical disorder (e.g., reduce the dementia onsets or detected dementia scores), and iii) predicting populations of patients that can be helped by applications of the learned treatments via specifically tailored or configured devices (e.g., providing a portable, wearable neuromodulation device).

Moreover, the disclosed framework is configured to not only detect symptoms of a medical disorder, but also conditions precedent (e.g., biomedical markers) that typically lead to specific disorders. This information can be fed to the systems and methods disclosed herein in order to enable it to detect the early onset of certain diseases before they occur (e.g., identify patients with fibromyalgia and predict their development risk of dementia).

According to some embodiments, a method is disclosed for dynamically adjusting and monitoring ML-controlled electronic stimulus patterns respective to a patient for purposes of diagnosing and treating the patient's medical conditions.

In accordance with one or more embodiments, the present disclosure provides a non-transitory computer-readable storage medium for carrying out the above mentioned technical steps. The non-transitory computer-readable storage medium has tangibly stored thereon, or tangibly encoded thereon, computer readable instructions that when executed by a device, cause at least one processor to perform a method for dynamically adjusting and monitoring ML-controlled electronic stimulus patterns respective to a patient for purposes of diagnosing and treating the patient's medical conditions.

In accordance with one or more embodiments, a system is provided that comprises one or more computing devices and/or apparatus configured to provide functionality in accordance with such embodiments. In accordance with one or more embodiments, functionality is embodied in steps of a method performed by at least one computing device and/or apparatus. In accordance with one or more embodiments, program code (or program logic) executed by a processor(s) of a computing device to implement functionality in accordance with one or more such embodiments is embodied in, by and/or on a non-transitory computer-readable medium.

### BRIEF DESCRIPTION OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIG. 3 illustrates an exemplary data flow according to some embodiments of the present disclosure;

FIG. 4 illustrates an exemplary data flow according to some embodiments of the present disclosure;

FIG. 5 illustrates an exemplary data flow according to some embodiments of the present disclosure;

FIG. 6 illustrates an exemplary data flow according to some embodiments of the present disclosure; and

FIG. 7 is a block diagram illustrating a computing device showing an example of a device used in various embodiments of the present disclosure.

### DETAILED DESCRIPTION OF EXAMPLE EMBODIMENTS

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as “up,” “down,” “bottom,” “top,” “front,” “rear,” “upper,” “lower,” “upwardly,” “downwardly,” and other orientational descriptors are intended to facilitate the description of the exemplary embodiments of the present disclosure, and are not intended to limit the structure of the exemplary embodiments of the present disclosure to any particular position or orientation. Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, wearable neuromodulation device, and the like, or some combination thereof.

A client device (UE) may vary in terms of capabilities or features. The disclosed (and claimed) subject matter is intended to cover a wide range of potential variations, such as a web-enabled client device or previously mentioned devices that may include a high-resolution screen (HD or 4K for example), one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

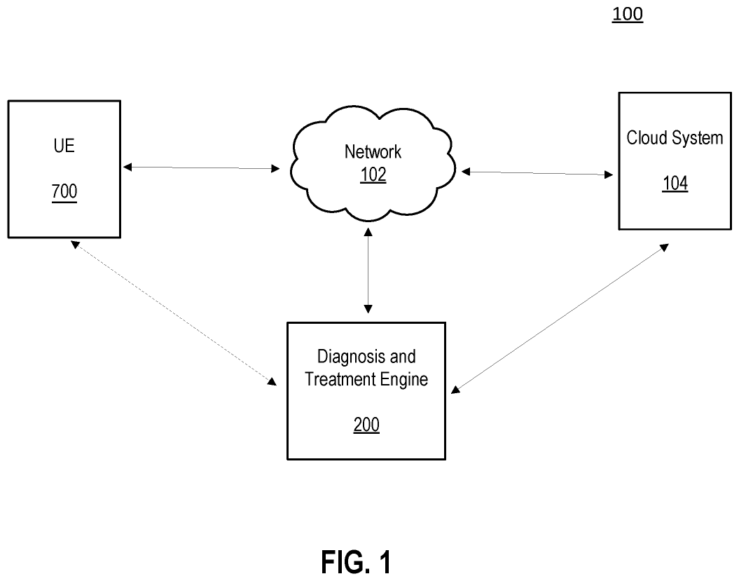

With reference to FIG. 1, system (or framework) 100 is depicted which includes UE 700 (e.g., a client device), network 102, cloud system 104 and diagnosis and treatment engine 200. UE 700 can be any type of device, such as, but not limited to, a mobile phone, tablet, laptop, personal computer, sensor, Internet of Things (IoT) device, autonomous machine, and any other device equipped with a cellular or wireless or wired transceiver.

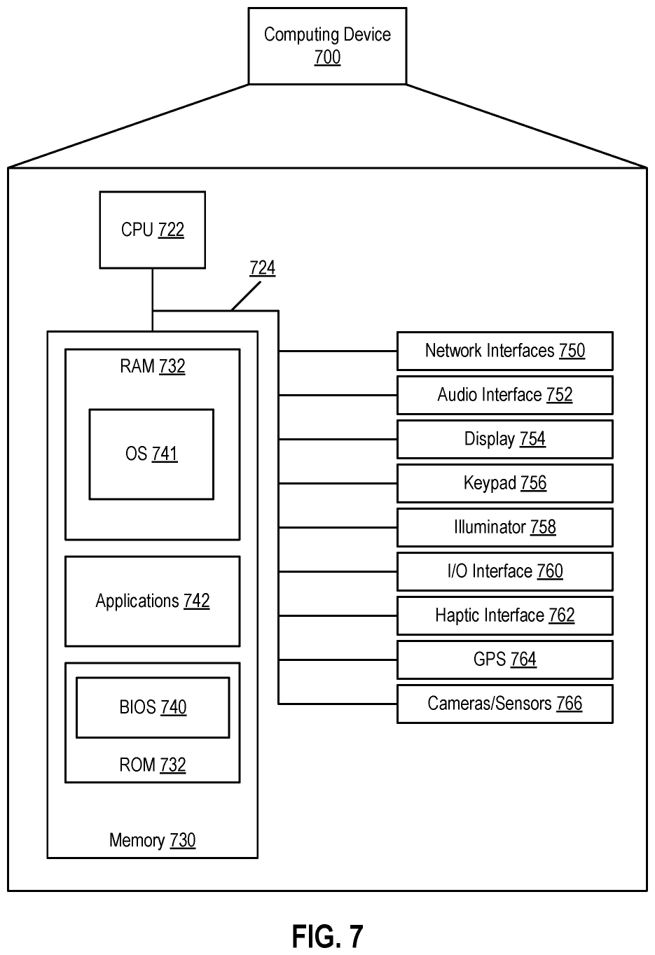

In some embodiments, as discussed above, UE 700 can also be a medical device, or another device that is communicatively coupled to a medical device that enables reception of readings from sensors of the medical device. For example, in some embodiments, UE 700 can be a wearable neuromodulation device. In another example, in some embodiments, UE 700 can be a user's smartphone that is connected via WiFi, Bluetooth Low Energy (BLE) or NFC, for example, to a peripheral neuromodulation device. Thus, in some embodiments, UE 700 can be configured to receive data from sensors associated with a medical device, as discussed in more detail below. Further discussion of UE 700 is provided below at least in reference to FIG. 7.

Network 102 can be any type of network, such as, but not limited to, a wireless network, cellular network, the Internet, and the like (as discussed above). As discussed herein, network 102 can facilitate connectivity of the components of system 100, as illustrated in FIG. 1.

Cloud system 104 can be any type of cloud operating platform and/or network based system upon which applications, operations, and/or other forms of network resources can be located. For example, system 104 can correspond to a service provider, network provider and/or medical provider from where services and/or applications can be accessed, sourced or executed from. In some embodiments, cloud system 104 can include a server(s) and/or a database of information which is accessible over network 102. In some embodiments, a database (not shown) of system 104 can store a dataset of data and metadata associated with local and/or network information related to a user(s) of UE 700, patients and the UE 700, and the services and applications provided by cloud system 104 and/or diagnosis and treatment engine 200.

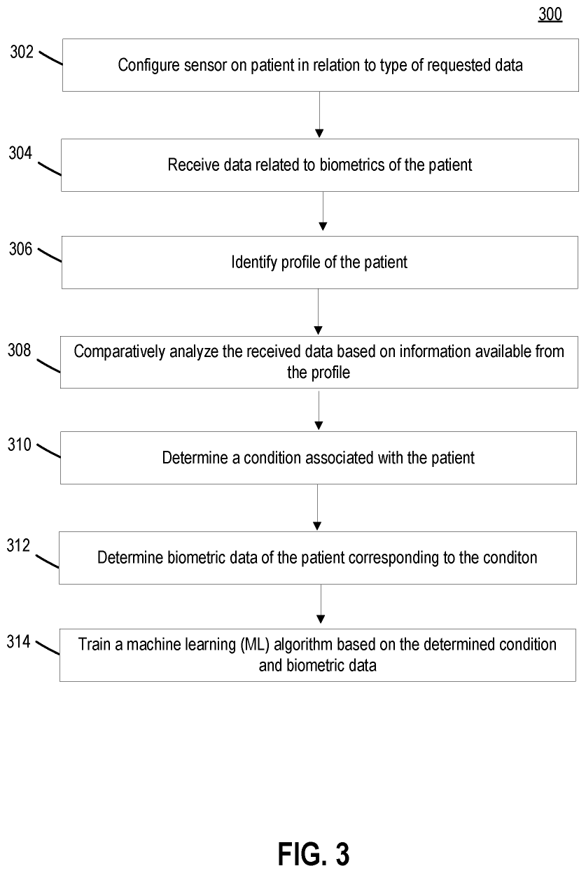

Diagnosis and treatment engine 200, as discussed below in more detail, includes components for determining diagnostics and treatment plans for a patient, and facilitating execution of such treatment plans. That is, for example, engine 200 can enable UE 700 to detect, receive or otherwise identify electronic data related to a patient, determine conditions therefrom by which a medical disorder can be determined, and then, upon determining a treatment plan, dynamically execute the treatment plan in a manner that accounts for characteristics of the patient and the iterative feedback received while treatments are being administered. Embodiments of how this is performed via engine 200, among others, are discussed in more detail below in relation to FIGS. 3-6.

According to some embodiments, diagnosis and treatment engine 200 can be a special purpose machine or processor and could be hosted by a device on network 102, within cloud system 104 and/or on UE 700. In some embodiments, engine 200 can be hosted by a peripheral device connected to UE 700 (e.g., a medical device, as discussed above).

According to some embodiments, diagnosis and treatment engine 200 can function as an application provided by cloud system 104. In some embodiments, engine 200 can function as an application installed on UE 700. In some embodiments, such application can be a web-based application accessed by UE 700 over network 102 from cloud system 104 (e.g., as indicated by the connection between network 102 and engine 200, and/or the dashed line between UE 700 and engine 200 in FIG. 1). In some embodiments, engine 200 can be configured and/or installed as an augmenting script, program or application (e.g., a plug-in or extension) to another application or program provided by cloud system 104 and/or executing on UE 700.

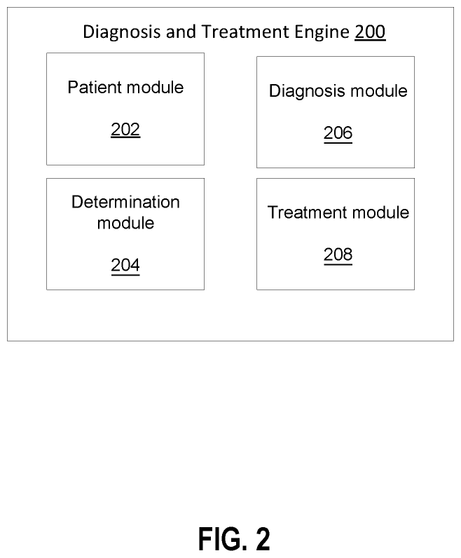

As illustrated in FIG. 2, according to some embodiments, diagnosis and treatment engine 200 includes patient module 202, determination module 204, diagnosis module 206 and treatment module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning now to FIGS. 3-6, disclosed are embodiments for a computerized framework that detects medical conditions within patients and dynamically effectuates a treatment via a recursively trained ML algorithm. According to some embodiments, the disclosed framework is configured for analyzing data associated with a patient, determining underlying conditions of the patient, then automatically causing medical equipment to output electronic stimuli that can address the medical condition(s) detected. As discussed herein, the disclosed framework can determine a correlation between a patient's attributes, electronic data of a condition of a patient and a medical disorder, and cause a device (e.g., a neuromodulation device) to treat and monitor improvements of the condition and disorder.

By way of a non-limiting example, the disclosed framework can operate by determining a correlation between slow wave sleep and dementia stage/progression. In some embodiments, the framework can function by operating a connected device (e.g., a wearable neuromodulation device) to detect slow wave sleep of a patient and monitor its effect on dementia with an aim of slowing its progression.

It is generally known by those in the medical community that fibromyalgia is debilitating condition associated with chronic pain, sleep disorders, mood disturbances and overall lowered quality of life. Fibromyalgia carries a 2.77 increased risk for developing dementia of any type. Reduced (or low) Heart Rate Variability (HRV) is a diagnostic signal for dementia, as this condition is associated with low vagal activity. As such, HRV may be used as a proxy signal for slow wave sleep disturbance as the amount of slow-wave sleep is correlated with HRV.

Thus, as discussed herein, the disclosed framework, via engine 200, can predict current and future severity of dementia in patients with fibromyalgia based on a HRV response to an adaptive closed loop audio-visual stimulation (AVS) program delivered by UE 500 (e.g., a medical device such as neuromodulation device, for example). As discussed below, engine 200 can utilize classifiers and/or predictive modelling to use a detected HRV response to the AVS program (as well as other factors, such as, but not limited to, EMRs) to identify undiscovered dementia in a patient, as well as to predict the development risk of dementia for those with fibromyalgia. Moreover, engine 200 can be adapted to provide AVS to potentially address the underlying low HRV, which can enable the improvement of the reduced HRV, thereby improving the chances of not developing dementia and/or delaying its onset within the patient.

It should be understood that while the disclosure herein will be discussed with reference to engine 200 executing a ML algorithm, technique, technology or mechanism, it should not be construed as limiting, as any type of trainable software, technology and/or executable computer/program-logic can be utilized in a similar manner without departing from the scope of the instant disclosure. For example, an algorithm can be any type of known or to be known, ML algorithm, artificial intelligence (AI) algorithm, greedy algorithm, recursive algorithm, and the like, or some combination thereof. Moreover, the ML algorithm can be any type of known or to be known support vector machine or logistic regression predictive modelling, and the like.

Turning to FIG. 3, Process 300 is disclosed which details non-limiting example embodiments for training engine 200 to detect a condition associated with a medical disorder based on received biometric data of a patient.

According to some embodiments, Steps 302-306 of Process 300 can be performed via patient module 202 of diagnosis and treatment engine 200; Steps 308-312 can be performed by determination module 204; and Step 314 can be performed by diagnosis module 206.

While the discussion herein related to Process 300 (as well as Processes 400-600) are being discussed in relation to an individual patient, it should be understood that the applicability of these processes can be extended to any number of patients (e.g., a small test group to a particular demographic, for example) without departing from the scope of the instant disclosure.

Process 300 begins with Step 302 where engine 200 is configured in connection with sensors related to a patient. In other words, a patient has sensors positioned proximate to them (or on them) so that biometric data be read.

As discussed herein, and understood by those of skill in the art, sensors can be used to capture, retrieve, observe, detect or otherwise identify biometric data related to a patient. The sensors can be associated with, connected to, mounted on and/or extend from a medical device, or some combination thereof. In some embodiments, the sensors correspond to optical measurement methods (e.g., a photoplethysmogram (PPG) sensors), electrodes (e.g., an EKG device) or can correspond to hardware and accompanying modules within a device (e.g., a smart watch that can monitor movements, rotation, pulse, and the like). According to some embodiments, the sensors can be associated with a neuromodulation device. While focus herein will be in reference to a general medical device, it should not be construed as limiting, as engine 200 can operate within a commercial off-the-shelf (COTS) device, as most are currently configured with sensors to monitor health of a person, and can also operate within specialized medical devices (e.g., a portable neuromodulation stimulator device, for example).

Having the sensors configured respective to the patient, Process 300 proceeds to Step 304 where data related to biometrics of the patient are received. According to some embodiments, the types of biometric data can correspond to a type of testing or monitored readings (or activity) that is being performed, can correspond to a type of medical device (and/or sensor) being used, and the like, or some combination thereof.

For purposes of this disclosure, the biometric data will be discussed with reference to collected HRV data for a patient from AVS sessions via a neuromodulation device in order to detect fibromyalgia for purposes of predicting and treating dementia.

In other words, the disclosure herein, via engine 200, provides for a closed-loop feedback algorithm that will read HRV signals in real time and modulate the AVS signal to optimize HRV response. The collection HRV signals, and modulation of the AVS signals enables the training of engine 200, and also enables engine 200 to optimize its implementation so as to treat the detected medical conditions, as discussed herein.

However, such operating embodiments should not be construed as limiting, as any type of known or to be known biometric data can be collected, any type of known or to be known medical device can be used, and/or any type of known or to be known medical condition can be identified via the disclosed systems and methods discussed herein (e.g., at least in FIGS. 3-6) without departing from the scope of the instant disclosure.

As such, for example, Process 300 (and Process 400-600) can involve engine 200 operating within environments to detect and manipulate electronic stimulus data related to, but not limited to, HRV data, EOG (electrooculography) data, EEG data, data from skin sensors, blood flow data, eye tracking data, data related to a hand(s) of a patient via virtual reality/augmented reality (VR/AR) controllers, muscle tension data, hemispheric data (e.g., balance) and the like, which can be leveraged for detecting and treating any type of known or to be known medical condition, such as, but not limited to, Alzheimer's disease, dementia, and the like.

In Step 306, profile data related to the patient is identified. The profile, as discussed above, can correspond to an EMR of the patient. As understood by those of skill in the art, an EMR for patient can include any type of patient information, including, but not limited to, an identity (ID), name, address, age, race, gender (and/or any other type of demographic or geographic information), medical history, prescription history, family medical history, insurance information, collected biometric data, and the like, or some combination thereof.

In Step 308, engine 200 then operates to comparatively analyze the received biometric data based on information available from the profile. According to some embodiments, Step 308 can involve parsing the profile to determine which information is relevant for the comparative analysis of Step 308. That is, for example, Step 308 can involve determining, deriving or otherwise identifying that the type of data collected in Step 304 corresponds to a testing for dementia (or other related conditions) based on the identification that the data is HRV data (e.g., a particular type). Based on this determination, engine 200 can then parse the patient's profile mining for sleep related or fibromyalgia related diagnostics (e.g., slow wave sleep information). Upon identifying this information from the profile, engine 200 can then comparatively analyze the received biometric data to the mined profile data.

According to some embodiments, the comparative analysis performed in Step 308 can involve the received biometric data and the mined profile data being used as inputs to any type of ML/AI computational analysis algorithm, technology, mechanism or classifier, such as, but not limited to, neural networks (e.g., artificial neural network analysis (ANN), convolutional neural network (CNN) analysis, and the like), computer vision, cluster analysis, data mining, Bayesian network analysis, Hidden Markov models, logical model and/or tree analysis, and the like.

As a result of the analysis performed in Step 308, engine 200 can determine a condition associated with the patient, as in Step 310. That is, engine 200 can determine that, based on the biometric data received in Step 304, and the indicators from the profile (EMR) of the patient identified in Step 308), that the patient is predicted to suffer from a determined condition. For example, based on collected HRV data (Step 304), and the medical history of the patient (Step 808), the patient can be determined to have fibromyalgia.

In Step 312, based on the determined condition from Step 310, engine 200 then determines the biometric data of the patient that corresponds to the condition. That is, engine 200 can analyze the biometric data and determine that certain portions and/or types of data (e.g., low HRV data, for example) that correspond to a certain type of medical condition (e.g., fibromyalgia, for example).

In Step 314, the ML algorithm associated with engine 200 is then trained based on the determination of Step 312. This enables engine 200 to detect medical conditions directly from received electronic stimulus (e.g., biometric data collected) for a patient. Therefore, the processing steps of Process 300 provide engine 200 with capabilities to detect a medical condition in patients when presented with similar types of biometric data.

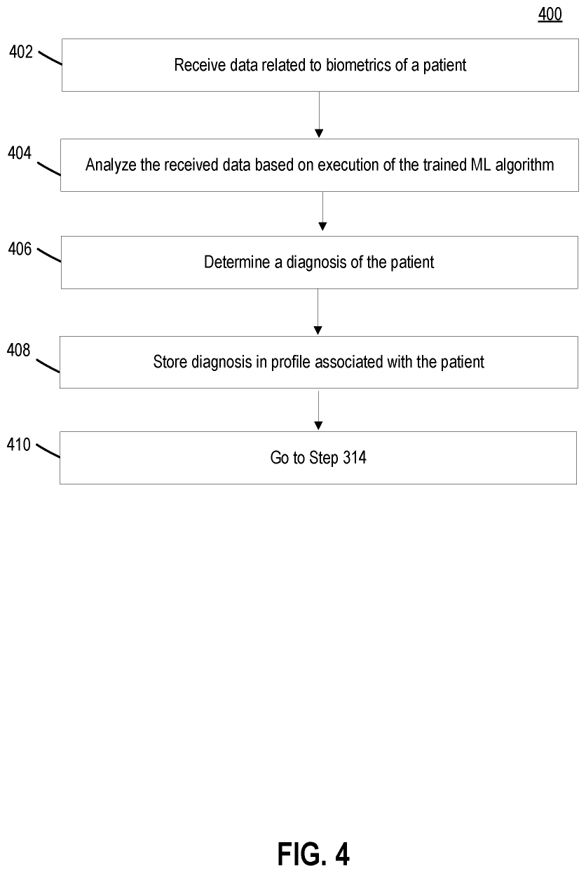

Turning to FIG. 4, Process 400 is disclosed which leverages the trained engine 200 (from Process 300, supra) to diagnosis a patient.

According to some embodiments, Steps 402-404 of Process 400 can be performed by patient module 202 of diagnosis and treatment module 200; Step 406 can be performed by determination module 204; and Steps 408-410 can be performed by diagnosis module 206.

Process 400 begins with Step 402 where data related to biometrics of a patient are received. Step 402 is performed in a similar manner as discussed above in relation to Steps 302-304, where sensors are positioned proximate (or on) a patient and electronic data is collected related to biometrics of the patient (e.g., HRV, for example).

In Step 404, the received data is analyzed via engine 200. According to some embodiments, engine 200 uses the received data from Step 402 as input data to the trained ML algorithm (from Step 314 of Process 300, supra). As mentioned above, the input data can be analyzed via the trained ML algorithm, which can be embodied as, but not limited to, a support vector machine, logistic regression predictive model, and the like.

In Step 406, as a result of the ML analysis of Step 404, engine 200 determines a diagnosis for the patient. The diagnosis is the output of engine 200, and can include information (or variables) related to, but not limited to, a detected medical condition(s), a medical disorder(s) or disease(s), prognosis of the disorder/disease (e.g., how advanced it is, or what stage or type), a correlation of the biometric data and how it maps to the prognosis, and the like, or some combination thereof.

For example, the diagnosis determined in Step 406 can indicate a range of HRV data for the patient, and an indication of how this maps a determined stage of fibromyalgia and type of dementia (that is expected in the future). In some embodiments, the diagnosis can also provide a predicted indicator related to when the onset of a predicted disease is to occur (e.g., in 2 years, with continued HRV levels in the same range, dementia will onset, for example).

In Step 408, information related to the determined diagnosis can be stored in the profile of the patient. For example, engine 200 can communicate with the entity housing the EMR of the patient (e.g., system 104 of FIG. 1), can cause the EMR hosted therein to be updated accordingly.

In some embodiments, the diagnosis can also or alternatively be stored in local memory associated with the medical device of the patient (e.g., the device used to detect the biometric data). For example, storage can occur in relation to a patient's personal neuromodulation, which enables the loading of diagnosis information for treating the condition in a more resource-friendly, streamlined manner.

According to some embodiments, this diagnosis information can also be used to further train the ML algorithm being executed by engine 200. Therefore, upon determining the diagnosis, this data can also be fed to engine 200 for further training (e.g., to provide additional information to train engine 200 based on the information of the patient(s) from Process 400), as in Step 410.

As such, Process 400 provides a non-limiting example embodiment where a patient can have a medical condition diagnosed via execution of the ML algorithm by engine 200 based on received biometric data of the patient.

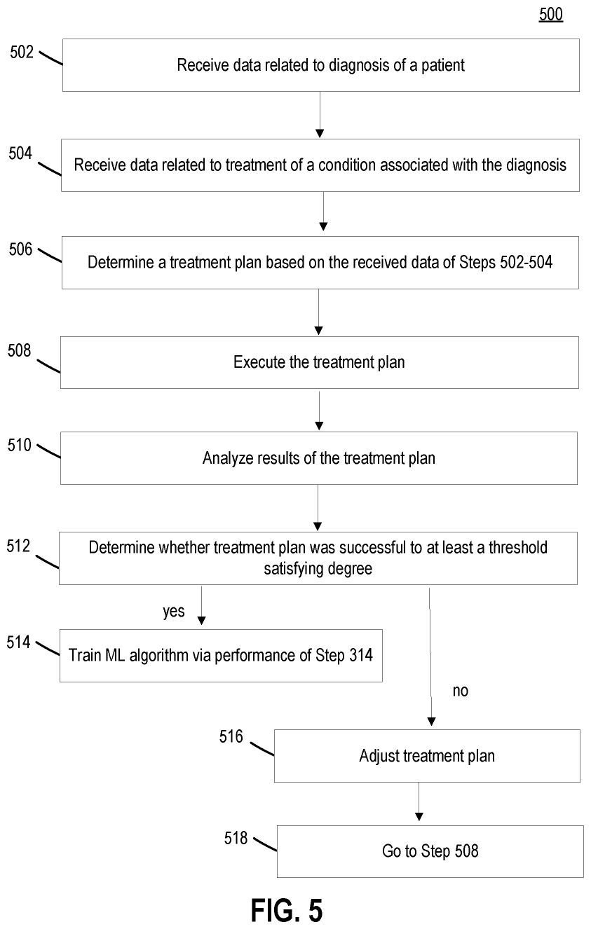

Turning to FIG. 5, Process 500 details non-limiting embodiments where engine 200 (and the ML algorithm executing therein) can be trained to determine and execute a treatment plan for a patient given a patient's determined diagnosis (for example, a diagnosis from Process 400 of FIG. 4, supra).

According to some embodiments, Steps 502-504 of Process 500 can be performed by patient module 202 of diagnosis and treatment module 200; Step 506 and 512 can be performed by determination module 204; Steps 508-510 and 514-518 can be performed by treatment module 208.

Process 500 begins with Step 502 where data related to the diagnosis of a patient is received. In some embodiments, this can involve retrieving diagnosis information from the EMR of the patient. In some embodiments, as discussed above, the diagnosis information of the patient may be stored on the medical device of the patient; therefore, Step 502 can involve retrieving such information from local memory.

In Step 504, engine 200 receives information related to treatment of the condition associated with the diagnosis. In other words, engine 200 can search for, receive or otherwise identify predetermined treatments for the type of condition indicated in the patient's diagnosis.

In Step 506, engine 200 determines a treatment plan for the patient based on the patient's diagnosis and the treatment data (from Steps 502 and 504, respectively). According to some embodiments, engine 200 can use the diagnosis data and the treatment data as input data to the ML algorithm (e.g., a support vector machine, logistic regression predictive model, as discussed above), whereby upon execution of the ML algorithm, engine 200 can determines a treatment plan specific for the patient (e.g., specific to the diagnosis).

According to some embodiments, the treatment plan can include information related to, but not limited to, value of electronic stimuli (e.g., AVS pattern) to output from a medical device (e.g., voltage, current, and the like), schedule for the output (e.g., frequency and length), a type of device to use to output the electronic stimuli, location of sensors on the patient to effectuate electronic stimuli, and the like, or some combination thereof.

In Step 508, engine 200 effectuates execution of the determined treatment plan. In some embodiments, engine 200 can control the medical device by providing instructions that cause the medical device to output electronic stimuli via sensors that correspond to the determined value, volume, rates, frequency (and the like) as dictated via the treatment plan.

In Step 510, in response to the applied treatment, results of the treatment plan are received and analyzed. In some embodiments, Step 510 can involve receiving response biometric data based on the treatment plan that can indicate how effective the treatment plan was (e.g., did the biometric markers associated with the condition/disease change). In some embodiments, Step 510 can be performed after a predetermined period of time (e.g., 2 weeks, for example, to enable enough treatments to occur so as to realize an impact, if any).

In Step 512, engine 200 can determine whether the treatment plan (determined in Step 508 and executed in Step 510) was successful (or effective). In some embodiments, Step 512 can involve determining whether the treatment plan alters the condition of the patient at least a threshold degree (or value). For example, if HRV values of the condition are at X, and after treatment the values change to Y, a determination is performed by engine 200 to discern whether Y satisfies the treatment threshold (e.g., is Y greater than threshold Z).

If the determination in Step 512 indicates that the treatment was successful to at least the threshold degree, then Process 500 proceeds to Step 514. In Step 514, the ML algorithm associated within engine 200 is trained in a similar manner as discussed above in relation to Step 314 of Process 300. In some embodiments, Step 514 involves training engine 200 to automatically treat a condition and/or disease of a patient based on a provided diagnosis (e.g., the patient has fibromyalgia; therefore, perform the learned treatment plan). In some embodiments, Step 514 involves storage of the treatment plan, in a similar manner as storage of the diagnosis, as discussed above.

In some embodiments, if the determination in Step 512 results in an indication that the treatment plan was not successful (e.g., does not satisfy the treatment threshold), then Process 500 can proceed from Step 512 to Step 516. In Step 516, engine 200 can adjust the treatment plan. In some embodiments, the adjustment can be based on a deviation or differential related to how (or to what degree) the treatment plan failed to satisfy the treatment threshold. In some embodiments, weights or other forms of adjustments can be applied to the treatment plan.

By way of a non-limiting example, if the data resultant from the treatment plan's execution indicates that the treatment plan is at least R below threshold Z, then a weight proportional to R can be determined and applied to the treatment plan (of Step 506).

After the adjustment of Step 516, Process recursively proceeds via Step 518 back to Step 508, where the adjusted treatment plan is executed. From there, Process 500 can recursively operate via Steps 508-518 until the ML algorithm is trained via Step 514.

As such, Process 500 provides a non-limiting example operating environment where engine 200 is trained to be able to automatically determine a treatment plan for a patient based on a provided diagnosis (which can be automatically determined via engine 200, via Process 400, supra).

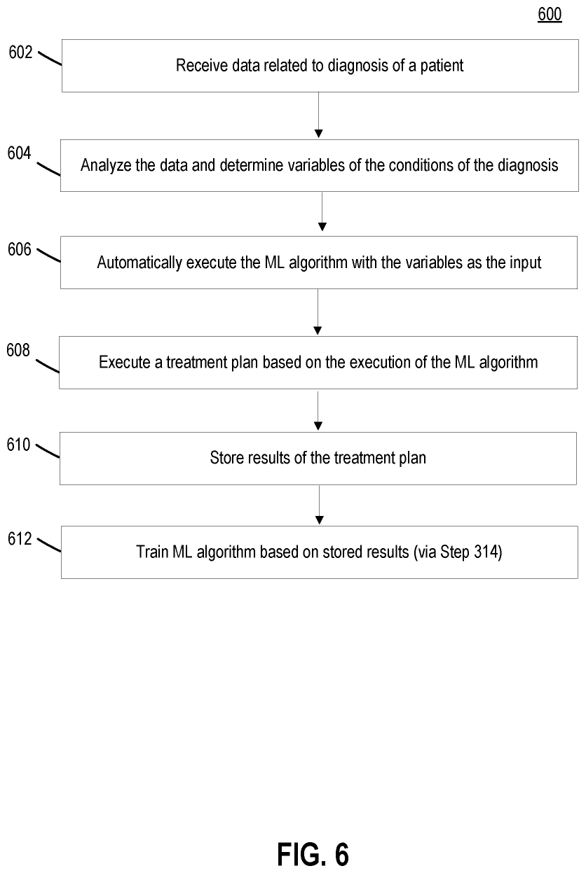

Turning to FIG. 6, Process 600 is disclosed which details non-limiting example embodiments for automatically performing the treatment diagnosis and execution according to the trained ML algorithm embodied engine 200.

According to some embodiments, Step 602 can be performed by patient module 202 of diagnosis and treatment module 200; Step 604 can be performed by determination module 204; and Steps 606-612 can be performed by treatment module 208.

Process 600 begins with Step 602 where data related to diagnosis of a patient is received. This can be performed in a similar manner as discussed above in relation to Step 502 of Process 500, supra.

In Step 604, engine 200 can analyze the diagnosis data and determines variables of the conditions of the diagnosis. This, as discussed above, can involve indicators related to a severity and/or stage of a diagnosed disease (see, e.g., Step 406, supra).

In Step 606, engine 200 executes the ML algorithm based on the identified variables, which enables the determination of a treatment plan. The treatment plan's determination, as well as the details of its anticipated/predicted efficacy and variables (e.g., schedule, frequency, and the like) are determined based on the learned ML algorithm being executed by engine 200.

In Step 608, the treatment plan is executed by engine 200. This can be performed in a similar manner as discussed above in relation to Step 508 of Process 500, supra. As such, a medical device is caused to output electronic stimuli as a means to predictively address (e.g., address or cure) a medical condition and/or disease. For example, the output stimuli can correspond to Vegas-nerve stimulation.

In Step 610, the results of the treatment plan are identified and stored. This can be performed in a similar manner as discussed above in relation to the storage of Step 408 of Process 400, supra.

And, in Step 612, the results from the executed treatment plan are fed back to the ML algorithm of engine 200 so as to train it further and improve its treatment plan predictive functionality. This can be performed in a similar manner as discussed above to the training of Step 314, supra.

Thus, Process 600 provides a non-limiting example embodiment of implementing a trained ML algorithm embodied engine 200 that can automatically determine and execute a treatment plan for a medical condition and/or disease provided for in a medical diagnosis.

FIG. 7 is a block diagram illustrating a computing device 700 (e.g., UE 700, as discussed above) showing an example of a client device or server device used in the various embodiments of the disclosure.

The computing device 700 may include more or fewer components than those shown in FIG. 7, depending on the deployment or usage of the device 700. For example, a server computing device, such as a rack-mounted server, may not include audio interfaces 752, displays 754, keypads 756, illuminators 758, haptic interfaces 762, GPS receivers 764, or cameras/sensors 766. Some devices may include additional components not shown, such as graphics processing unit (GPU) devices, cryptographic co-processors, AI accelerators, or other peripheral devices.

As shown in FIG. 7, the device 700 includes a central processing unit (CPU) 722 in communication with a mass memory 730 via a bus 724. The computing device 700 also includes one or more network interfaces 750, an audio interface 752, a display 754, a keypad 756, an illuminator 758, an input/output interface 760, a haptic interface 762, an optional GPS receiver 764 (and/or an interchangeable or additional GNSS receiver) and a camera(s) or other optical, thermal, or electromagnetic sensors 766. Device 700 can include one camera/sensor 766 or a plurality of cameras/sensors 766. The positioning of the camera(s)/sensor(s) 766 on the device 700 can change per device 700 model, per device 700 capabilities, and the like, or some combination thereof.

In some embodiments, the CPU 722 may comprise a general-purpose CPU. The CPU 722 may comprise a single-core or multiple-core CPU. The CPU 722 may comprise a system-on-a-chip (SoC) or a similar embedded system. In some embodiments, a GPU may be used in place of, or in combination with, a CPU 722. Mass memory 730 may comprise a dynamic random-access memory (DRAM) device, a static random-access memory device (SRAM), or a Flash (e.g., NAND Flash) memory device. In some embodiments, mass memory 730 may comprise a combination of such memory types. In one embodiment, the bus 724 may comprise a Peripheral Component Interconnect Express (PCIe) bus. In some embodiments, the bus 724 may comprise multiple busses instead of a single bus.

Mass memory 730 illustrates another example of computer storage media for the storage of information such as computer-readable instructions, data structures, program modules, or other data. Mass memory 730 stores a basic input/output system (“BIOS”) 740 for controlling the low-level operation of the computing device 700. The mass memory also stores an operating system 741 for controlling the operation of the computing device 700.

Applications 742 may include computer-executable instructions which, when executed by the computing device 700, perform any of the methods (or portions of the methods) described previously in the description of the preceding Figures. In some embodiments, the software or programs implementing the method embodiments can be read from a hard disk drive (not illustrated) and temporarily stored in RAM 732 by CPU 722. CPU 722 may then read the software or data from RAM 732, process them, and store them to RAM 732 again.

The computing device 700 may optionally communicate with a base station (not shown) or directly with another computing device. Network interface 750 is sometimes known as a transceiver, transceiving device, or network interface card (NIC).

The audio interface 752 produces and receives audio signals such as the sound of a human voice. For example, the audio interface 752 may be coupled to a speaker and microphone (not shown) to enable telecommunication with others or generate an audio acknowledgment for some action. Display 754 may be a liquid crystal display (LCD), gas plasma, light-emitting diode (LED), or any other type of display used with a computing device. Display 754 may also include a touch-sensitive screen arranged to receive input from an object such as a stylus or a digit from a human hand.

Keypad 756 may comprise any input device arranged to receive input from a user. Illuminator 758 may provide a status indication or provide light.

The computing device 700 also comprises an input/output interface 760 for communicating with external devices, using communication technologies, such as USB, infrared, Bluetooth™, or the like. The haptic interface 762 provides tactile feedback to a user of the client device.

The optional GPS transceiver 764 can determine the physical coordinates of the computing device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS, or the like, to further determine the physical location of the computing device 700 on the surface of the Earth. In one embodiment, however, the computing device 700 may communicate through other components, provide other information that may be employed to determine a physical location of the device, including, for example, a MAC address, IP address, or the like.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

For the purposes of this disclosure the term “user”, “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising the steps of:
receiving, by a device, biometric data related to a patient, the biometric data corresponding to monitored readings collected by sensors associated with the device;
analyzing, by the device, the received biometric data, and determining, based on the analysis, a diagnosis of the patient, the diagnosis corresponding to a medical condition of the patient;
determining, by the device, based on determined diagnosis, a treatment plan for the patient, the treatment plan comprising instructions that correspond to the medical condition; and
executing, by the device, the treatment plan, the execution comprising automatically communicating electronic stimuli via the sensors to the patient.

2. The method of claim 1, further comprising:
identifying, by the device, a set of biometric data related to a set of patients;
identifying, by the device, electronic medical records (EMRs) for each patient in the set of patients;
performing, by the device, comparative analysis of the set of biometric data and the EMRs;
determining, by the device, a medical condition for each patient in the set of patients;
determining, by the device, portions of the set of biometric data that correspond to the determined medical conditions; and
training, by the device, a machine learning (ML) engine based on the determined portions of the biometric data.

3. The method of claim 2, wherein the determination of the diagnosis of the patient is performed via the device executing the ML engine.

4. The method of claim 1, further comprising:
identifying, by the device, a diagnosis for a set of patients that corresponds to a medical condition;
identifying, by the device, a treatment for the diagnosis for the set of patients;
determining, by the device, a treatment plan for the set of patients;
executing, by the device, the treatment plan;
analyzing, by the device, results of the treatment plan on each of the set of patients; and
determining, by the device, whether the treatment plan was effective against the medical condition for the set of patients.

5. The method of claim 4, further comprising:
training, by the device, a machine learning (ML) engine based on the treatment plan when the determination indicates the treatment plan was effective, wherein the determination of the treatment plan is performed via the device executing the ML engine.

6. The method of claim 4, further comprising:
adjusting, by the device, the treatment plan when the determination indicates that the treatment plan was not effective; and
executing, by the device, the adjusted treatment plan, wherein a machine learning (ML) engine is trained when the adjusted treatment plan is determined to be effective against the medical condition for the set of patients.

7. The method of claim 1, wherein the diagnosis comprises information related to at least one of a medical disorder disease related to the medical condition, a prognosis of the medical disease, and a correlation of the biometric data and its mapping to the prognosis.

8. The method of claim 1, wherein the treatment plan comprises information related to at least one of a value of the electronic stimuli to output from the device, a schedule for the output, a type of device to use to output the electronic stimuli, and a location of the sensors on the patient to effectuate electronic stimuli.

9. The method of claim 1, further comprising:
communicating, by the device, an adaptive closed loop audio-visual stimulation (AVS) program, wherein the biometric data is received in response to the transmitted AVS program.

10. The method of claim 9, wherein the transmitted electronic stimuli correspond to the AVS program.

11. The method of claim 1, wherein the steps are performed by the device executing at least one of a support vector machine or logistic regression predictive modelling algorithm.

12. The method of claim 1, wherein the device is associated with a wearable neuromodulation device.

13. A non-transitory computer-readable storage medium tangibly encoded with computer-executable instructions, that when executed by a device, perform a method comprising steps of:
receiving, by the device, biometric data related to a patient, the biometric data corresponding to monitored readings collected by sensors associated with the device;
analyzing, by the device, the received biometric data, and determining, based on the analysis, a diagnosis of the patient, the diagnosis corresponding to a medical condition of the patient;
determining, by the device, based on determined diagnosis, a treatment plan for the patient, the treatment plan comprising instructions that correspond to the medical condition; and
executing, by the device, the treatment plan, the execution comprising automatically communicating electronic stimuli via the sensors to the patient.

14. The non-transitory computer-readable storage medium of claim 13, further comprising:
identifying, by the device, a set of biometric data related to a set of patients;
identifying, by the device, electronic medical records (EMRs) for each patient in the set of patients;
performing, by the device, comparative analysis of the set of biometric data and the EMRs;
determining, by the device, a medical condition for each patient in the set of patients;
determining, by the device, portions of the set of biometric data that correspond to the determined medical conditions; and
training, by the device, a machine learning (ML) engine based on the determined portions of the biometric data, wherein the determination of the diagnosis of the patient is performed via the device executing the ML engine.

15. The non-transitory computer-readable storage medium of claim 13, further comprising:
identifying, by the device, a diagnosis for a set of patients that corresponds to a medical condition;
identifying, by the device, a treatment for the diagnosis for the set of patients;
determining, by the device, a treatment plan for the set of patients;
executing, by the device, the treatment plan;
analyzing, by the device, results of the treatment plan on each of the set of patients; and
determining, by the device, whether the treatment plan was effective against the medical condition for the set of patients.

16. The non-transitory computer-readable storage medium of claim 15, further comprising:
when the determination indicates the treatment plan was effective:
training, by the device, a machine learning (ML) engine based on the treatment plan, wherein the determination of the treatment plan is performed via the device executing the ML engine; and
when the determination indicates that the treatment plan was not effective:
adjusting, by the device, the treatment plan; and
executing, by the device, the adjusted treatment plan, wherein a machine learning (ML) engine is trained when the adjusted treatment plan is determined to be effective against the medical condition for the set of patients.

17. A device comprising:
a processor configured to:
receive biometric data related to a patient, the biometric data corresponding to monitored readings collected by sensors associated with the device;
analyze the received biometric data, and determine, based on the analysis, a diagnosis of the patient, the diagnosis corresponding to a medical condition of the patient;
determine, based on determined diagnosis, a treatment plan for the patient, the treatment plan comprising instructions that correspond to the medical condition; and
execute the treatment plan, the execution comprising automatically communicating electronic stimuli via the sensors to the patient.

18. The device of claim 17, wherein the processor is further configured to:
identify a set of biometric data related to a set of patients;
identify electronic medical records (EMRs) for each patient in the set of patients;
perform comparative analysis of the set of biometric data and the EMRs;
determine a medical condition for each patient in the set of patients;
determine portions of the set of biometric data that correspond to the determined medical conditions; and
train a machine learning (ML) engine based on the determined portions of the biometric data, wherein the determination of the diagnosis of the patient is performed via the device executing the ML engine.

19. The device of claim 17, wherein the processor is further configured to:
identify a diagnosis for a set of patients that corresponds to a medical condition;
identify a treatment for the diagnosis for the set of patients;
determine a treatment plan for the set of patients;
execute the treatment plan;
analyze results of the treatment plan on each of the set of patients; and
determine whether the treatment plan was effective against the medical condition for the set of patients.

20. The device of claim 19, wherein the processor is further configured to:
when the determination indicates the treatment plan was effective:
train a machine learning (ML) engine based on the treatment plan, wherein the determination of the treatment plan is performed via the device executing the ML engine; and
when the determination indicates that the treatment plan was not effective:
adjust the treatment plan; and
execute the adjusted treatment plan, wherein a machine learning (ML) engine is trained when the adjusted treatment plan is determined to be effective against the medical condition for the set of patients.
